(19) United States
(12) Patent Application Publication
(10) Pub. No.: US 2002/0183265 A1
(43) Pub. Date: Dec. 5, 2002

(54) Manufacture and use of an antibiotic/antibiotics preparation

(75) Inventors: Sebastian Vogt, Jena (DE); Matthias Schnabelrauch, Jena (DE); Klaus-Dieter Kuhn, Marburg (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(73) Assignee: Heraues Kulzer GmbH & Co.KG, Hanau (DE)

(21) Appl. No.: 10/101,254

(22) Filed: Mar. 19, 2002

(30) Foreign Application Priority Data
Mar. 22, 2001 (DE) 101 14 245.5

(51) Int. Cl. A61K 31/7048; A61K 31/7008; B27N 3/00; A61K 31/65; A61K 31/47; A61K 31/496

(52) U.S. Cl. 514/29; 514/152; 514/253.08; 514/312; 264/109

(57) ABSTRACT

The present invention concerns the manufacture and use of an antibiotic/antibiotics preparation for human and veterinary medicine, for the treatment of local microbial infections in hard and soft tissue. The manufacture in accordance with the invention of an antibiotic/antibiotics preparation takes place according to the invention in that water, an amphiphilic component of a representative of the alkyl sulfates, aryl sulfates, alkylaryl sulfates, cycloalkyl sulfates, alkylcycloalkyl sulfates, alkyl sulfamates, cycloalkyl sulfamates, alkylcycloalkyl sulfamates, aryl sulfamates, alkylaryl sulfamates, alkyl sulfonates, fatty acid-2-sulfonates, aryl sulfonates, alkylaryl sulfonates, cycloalkyl sulfonates, cycloalkyl sulfonates, alkylcycloalkyl sulfonates, alkyl disulfates, cycloalkyl disulfates, alkyl disulfonates, cycloalkyl disulfonates, aryl disulfonates, alkylaryl disulfonates, aryl trisulfonates and alkylaryl trisulfonates, one or more antibiotic components from the group of aminoglycoside antibiotics, lincosamide antibiotics and tetracycline antibiotics, an organic auxiliary component and/or an inorganic auxiliary component and if need be at least one biologically active component are mixed together and shaped into molded elements, granulates, powders, foils, shaped masses and threads.

The present invention concerns the manufacture and use of an antibiotic/antibiotics preparation with delayed active ingredient release for human and veterinary medicine for the treatment of local microbial infections in hard and soft tissues.

The treatment of local microbial infections of hard and soft tissue in human and veterinary medicine requires high local antibiotics concentrations in the infected tissue region. It has been known for a long time that a systemic application of antibiotics is encumbered by a series of problems. With systemic use, it is often necessary to use very high antibiotics doses so that antimicrobially effective antibiotics concentrations are attained in the infected tissue. In this way, severe damage to the organism can occur in particular with aminoglucoside antibiotics and with tetracycline type antibiotics owing to their nephrotoxicity and ototoxicity. Therefore the idea of using antibiotics in locally applicable release systems or transferring them in suitable deposit forms suggested itself.

Deposit systems for delayed release of antibiotics for the treatment of local infections are the object of a great number of publications and patents. These can generally be classified according to two fundamental retarding mechanisms. The one action principle consists of the physical fixation of the antibiotics through adsorption to a matrix or through inclusion in a non-resorbable or resorbable matrix. The second chemical delay principle consists of using sparingly soluble antibiotic salts which dissolve slowly following appropriate application in the human or animal organism while active ingredients are being released.

The physical fixation of antibiotics while using non-resorbable plastics was the content of a series of patents of which here only a few are being presented as examples. Thus Klemm (K. Klemm, Surgical synthetic resin material and method of treating osteomyelitis. May 13, 1975, U.S. Pat. No. 3,882,858) proposes treating osteomyelitis with plastic particles of polymethacrylate, polyacrylate or their copolymers which are impregnated with gentamicin (or: gentamycin) or other antibiotics. Klemm describes the use of septopal (K. Klemm: Septopal—a new way of local antibiotic therapy. In T. J. G. Van Rens, F. H. Kayser, (eds), Local Antibiotic Treatment in Osteomyelitis and Soft Tissue Infections, Excerpta Medica, Amsterdam (1981) 24-31; K. Klemm: Antibiotic beat chains. Clin. Orthop. Relat. Res. 295 (1993) 63-76). These are commercially available gentamycin-releasing chains of polymethacrylate. Heuser and Dingeldein describe a composition on the basis of antibiotics and polymethymethacrylate or polyacrylate to which amino acids are added as additional components (D. Heuser, E. Dingeldein: Synthetic resin-base, antibiotic compositons containing amino acids. Apr. 4, 1980, U.S. Pat. No. 4, 191,740; D. Heuser, E. Dingeldein: Synthetic resin-base antibiotic compositions containing amino acids. Nov. 11, 1980, U.S. Pat. No. 4,233,287). Furthermore, antibiotics, especially aminoglycoside antibiotics, were incorporated into bone cements (A. Gross, R. Schaefer, S. Reiss: Bone cement compositions containing gentamycin. Nov. 22, 1977, U.S. Pat. No. 4,059,684; A. Welch: Antibiotics in acrylic bone cement. In vitro studies. J. Biomed. Mater. Res. 12 (1978) 679; R. A. Elson, A. E. Jephott, D. B. McGechie, D. Vereitas: Antibiotic-loaded acrylic cement. J. Bone Joint Surg. 59B (1977) 200-205.

The physical fixation of antibiotics with the aid of resorbable plastics, especially of polyesters of α-hydroxy carboxylic acids, was likewise the object of a series of publications, of which only a few are reported here by way of example. Sampath et al. propose a gentamycin-releasing system consisting of poly-L-lactide and gentamycin which was manufactured by the pressing of poly-L-lactide/gentamycin microcapsules (S. S. Sampath, K. Garvin, D. H. Robinson: Preparation and characterization of biodegradable poly(-L-lactic acid) gentamycin delivery systems. Int. J. Pharmaceutics 78 (1992) 165-174). This system shows, as a function of the amount of gentamycin used, a considerable delay in active substance release. In a similar system, poly-D,L-lactide was used for the manufacture of active ingredient-containing microspheres (R. Bodmeier, J. W. McGinity: The preparation and evaluation of drug-containing poly(D,L-lactide) microspheres formed by solvent evaporation method. Pharm. Res. 4 (1987) 465-471). Microparticles of poly lactide which are coated with collagen/gentamycin sulfate are likewise described by Fries and Schlapp (W. Fries, M. Schlapp: Advanced implants for local delivery of gentamicin. Sixth World Biomaterials Congress Transactions (2000) 1488). These coated microspheres showed but a very slight tendency to delay the release of gentamicin. Gentamicin-containing resorbable molded elements were proposed by Schmidt et. al. (C. Schmidt, R. Wenz, B. Nies, F. Moll: Antibiotic in vivo/in vitro release, histocompatibility and biodegradation of gentamicin implants based on lactic acid polymers and copolymers. J. Control. Release 37 (1995) 83-94). These elements were manufactured by the pressing of mixtures of gentamicin sulfate/poly-L-lactide, gentamicin sulfate/poly-D,L-lactide and gentamicin sulfate/poly-D,L-lactide-coglycolide. The deposit preparations released approximately ninety-percent of the antibiotic within twenty-four hours.

In addition to the physical fixation of antibiotics using plastics, numerous inorganic systems with retarding action were also described. Below only a few systems produced with calcium sulfate are reported by way of example. Thus a retarding system is described by Randolph et al., which is based upon the inclusion of active ingredients in a calcium sulfate matrix (D. A. Randolph, J. L. Negri, T. R. Devine, S. Gitelis: Calcium sulfate controlled release matrix. Sep. 15, 1998, U.S. Pat. No. 5,807,567). The manufacture of these calcium sulfate pellets takes place here proceeding from a mixture of α-calcium sulfate hemihydrate, β-calcium sulfate hemihydrate, an additive and water. Hardening takes place through the formation of calcium sulfate dihydrate. Turner et al. describe tablets of calcium sulfate which contain tobramycin and which are to be used to treat medullary defects (T. M. Turner, R. M. Urban, S. Gitelis, A. M. Lawrence-Smith, D. J. Hall: Delivery of tobramycin using calcium sulfate tablets to graft a large medullary defect: Local and systemic effects. Sixth World Biomaterials Congress Transactions (2000) 767). Similar release systems from calcium sulfate, but with amikacin sulfate, were likewise described (D. W. Petersen, W. O. Haggaard, L. H. Morris, K. C. Richelsoph, J. E. Parr: Elution of amikacin from calcium sulfate pellets: An in vitro study. Sixth World Biomaterials Congress Transactions (2000) 767).

Previously, sparingly soluble salts of aminoglycoside antibiotics and lincosamide antibiotics received relatively little attention for the manufacture of deposit preparations. The formation of hard to dissolve salts or chelates of antibiotics of the tetracycline type has been the general state of knowledge for years. Thus Folch Vazquez describes the manufacture of tetracycline dodecyl sulfate by the transformation of tetracycline hydrochloride with sodium dodecyl sulfate in water (C. Folch Vazquez: Tetracycline lauryl sulfate. Feb. 8, 1966, ES 3,309,402; C. Folch Vazquez: Tetracycline derivatives. Jan. 9, 1967, NL 6609490).

Alternatively, the manufacture can also take place proceeding from tetracycline and dodecyl sulfuric acid (C. Folch Vazquez: Tetracycline lauryl sulfate. Feb. 8, 1966, ES 322,771). Furthermore, the use of tetracycline sulfamates for antibiotic therapy was proposed (A. Jurando, J. M. Puigmarti: Antibiotic tetracycline sulfamate and its derivatives. Oct. 27, 1970, U.S. Pat. No. 3,536,759; Anonymous: Antibiotic tetracycline alkylsulfamates. Oct. 16, 1969, ES 354,173; C. Ciuro, A. Jurado: Stability of a tetracycline derivative. Afinidad 28 (292) 1971, 1333-5). A series of sparingly soluble salts is also basically known in connection with aminoglucoside antibiotics. Thus, with gentamicin, the synthesis of hard to dissolve salts based on higher fatty acids, aryl alkyl carboxylic acids, alkyl sulfates and alkyl sulfonates was described (G. M. Luedemann, M. J. Weinstein: Gentamycin and method of production. Jul. 16, 1962, U.S. Pat. No. 3,091,572). Gentamicin salts of lauric acid, stearic acid, palmitic acid, oleic acid or phenyl butyric acid, naphthalene-1-carboxylic acid, lauryl sulfuric acid and dodecylbenzenesulfonic acid are examples of this. These salts prove disadvantageous in many ways because they represent wax-like (or resinous), hydrophobic substances which impede a Galenic use. Despite this, fatty acid salts of gentamicin and etamycin were synthesized from the free base or from their salts in water at 50-80 C (H. Voege, P. Stadler, H. J. Zeiler, S. Samann, K. G. Metzger: Sparingly-soluble salts of aminoglycosides and formations containing them with inhibited substance release. Dec. 28, 1982, DE 3,248,328). These antibiotics-fatty acid salts are supposed to be suitable as injection preparations. The manufacture of gentamicin dodecyl sulfate and its use in salves (or ointments), cremes was likewise described (C. Folch Vazquez: Gentamicin derivatives. Oct. 29, 1974, BE 821,600). Sparingly soluble aminoglycoside flavonoid phosphates represent a more recent development (H. Wahlig, E. Dingeldein, R. Kirchlechner, D. Orth, W. Rogalski: Flavonoid phosphate salts of aminoglycoside antibiotics. Oct. 13, 1986, U.S. Pat. No. 4,617,293). The salts of phosphoric acid semi-esters of derivatives of hydroxy flavanes, hydroxy flavenes, hydroxy flavanones, hydroxy flavones and hydroxy flavylium are described. The derivatives of flavanones and flavones are especially preferred. The sparingly soluble salts are supposed to be used as deposit preparations. Thus, for example, the salts are used in collagen shaped mass (H. Wahlig, E. Dingeldein, D. Braun: Medicinally useful, shaped mass of collagen resorbable in the body. Sep. 22, 1981, U.S. Pat. No. 4,291,013). Furthermore, even artificial heart valves are impregnated with these sparingly soluble gentamicin salts, gentamicin crobefate (M. Cimbollek, B. Nies, R. Wenz, J. Kreuter: Antibiotic-impregnated heart valve sewing rings for treatment and prophylaxis of bacterial endocarditis. Antimicrob. Agents Chemother. 40(6) (1996) 1432-1437). With this patent, it is particularly interesting that a mixture of easily soluble gentamicin sulfate and sparingly soluble gentamicin crobefate is used.

The goal here was that, on the one hand, after introducing the heart valve rings into the organism or in a model fluid, a high initial gentamicin concentration is reached by the easily soluble gentamicin sulfate, and on the other hand through the relatively sparingly soluble gentamicin crobefate, a release of gentamicin over a longer period of time becomes possible. This means that the time-dependent release of gentamicin is controlled by the proportion of easily soluble gentamicin sulfate and sparingly soluble gentamicin crobefate. For a selective adjustment of the releasing behavior it is therefore necessary to use the two gentamicin salts in defined proportions in the Galenic formulations. This method of deposit formation through the combination of an easily soluble antibiotic salt with a sparingly soluble antibiotic salt presupposes the availability of a pure sparingly soluble salt form of an antibiotic.

In sum, it can be stated that the known antibiotic deposit systems with physically caused delay of the antibiotic release depend to a great extent on the composition and structure of the matrix used. Furthermore, the production process of these antibiotic systems is of considerable influence for the releasing behavior. The use of sparingly soluble antibiotic salts appears mainly to open the possibility of creating largely matrix-independent retarding- systems, as the patent U.S. Pat. No. 4,617,293 shows. The previous disadvantage of these systems consists in that, for each antibiotic from the aminoglycoside-antibiotics, the lincosamide antibiotics and the tetracycline antibiotics group used, a special form of salt must be synthesized prior to manufacture of the deposit preparation.

Underlying the present invention is the objective of creating a process for the manufacture of an antibiotic/antibiotics preparation with retarding active ingredient release for the treatment of local microbial infections in bone and soft tissue for human and veterinary medicine which overcomes the disadvantages of the known retarding antibiotic formulations. Sought is an antibiotic/antibiotics preparation which enables a controlled antibiotics release in a period of time up to approximately three weeks. The mechanism of delayed active ingredient release should basically be independent of the supporting material and should not rest upon adsorption effects on surfaces of the supporting materials. Sought is an antibiotic/antibiotics preparation which can be processed into implants while retaining active ingredient retardation with resorbable as well as non-resorbable auxiliary materials of the most varied structure. Furthermore, the method of antibiotic/antibiotics preparation should not only be applicable for a specific antibiotic, but rather it should be suited for a series of antibiotics of similar structure.

Underlying the invention is the surprising finding that a mixture of water, at least one amphiphilic component of a representative of the alkyl sulfates, aryl sulfates, alkylaryl sulfates, cycloalkyl sulfates, alkylcycloalkyl sulfates, alkyl sulfamates, cycloalkyl sulfamates, alkylcycloalkyl sulfamates, aryl sulfamates, alkylaryl sulfamates, alkyl sulfonates, fatty acid 2-sulfonates, aryl sulfonates, alkylaryl sulfonates, cycloalkyl sulfonates, alkylcycloalkyl sulfonates, alkyl disulfates, cycloalkyl disulfates, alkyl disulfonates, cycloalkyl disulfonates, aryl disulfonates, alkylaryl disulfonates, aryl trisulfonates and alkylaryl trisulfonates as well as at least one antibiotic component from the group of aminoglycoside antibiotics, lincosamide antibiotics and tetracycline antibiotics, an organic and/or an inorganic auxiliary component (or adjuvant) and if need be at least one biologically active auxiliary component yields an active ingredient formulation which can be processed into molded elements and/or granulates and/or powders and/or foils and/or shaped masses and/or threads, preferably by means of pressing and/or extrusion and/or grinding and/or calendering and/or casting and/or spinning and/or sintering. Surprisingly, these molded elements and coatings manifest a delayed antibiotics release over a period of time from several day up to several weeks following introduction to an aqueous medium.

The following embodiments have proven especially advantageous in practice.

In accordance with the invention, it is advantageous for the amphiphilic components from the alkyl sulfates, aryl sulfates, alkylaryl sulfates, cycloalkyl sulfates and alkylcycloalkyl sulfates group as semi-esters to be present in the form of a sodium salt and/or potassium salt and/or ammonium salt and/or trialkyl ammonium salt and/or dialkyl ammonium salt and/or monoalkyl ammonium salt and/or triaryl ammonium salt and/or diaryl ammonium salt and/or aryl ammonium salt and/or alkyldiaryl ammonium salt and/or dialkylaryl ammonium salt and/or tricycloalkyl ammonium salt and/or dicycloalkyl ammonium salt and/or monocycloalkyl ammonium salt and/or alkyldicycloalkyl ammonium salt and/or dialkylcycloalkyl ammonium salt and or in the form of an acid or an anhydride.

Furthermore, it is advantageous in accordance with the invention that the amphiphilic component from the alkyl sulfonates, fatty acid 2-sulfonates, alkyl sulfamates, cycloalkyl sulfamates, aryl sulfamates, alkylaryl sulfamates, aryl sulfonates, alkylaryl sulfonates, cycloalkyl sulfonates, alkylcycloalkyl sulfonates, alkyl disulfates, cycloalkyl disulfates, alkyl disulfonates, cycloalkyl disulfonates, aryl disulfonates, alkylaryl disulfonates, aryl trisulfonates and alkylaryl trisulfonates group are present in the form of a sodium salt and/or in the form of a potassium salt and/or in the form of an ammonium salt and/or in the form of a trialkyl ammonium salt and/or in the form of a dialkyl ammonium salt and/or in the form of a monoalkyl ammonium salt and/or in the form of a triaryl ammonium salt and/or in the form of a diaryl ammonium salt and/or in the form of an aryl ammonium salt and/or in the form of an alkyldiaryl ammonium salt and/or in the form of a dialkylaryl ammonium salt and/or in the form of a tricycloalkyl ammonium salt and/or in the form of a dicycloalkyl ammonium salt and/or in the form of a monocycloalkylammonium salt and/or in the form of an alkyldicycloalkyl ammonium salt and/or dialkylcycloalkyl ammonium salt and/or in the form of an sulfonic acid and/or in the form of a sulfonic acid anhydride.

In accordance with the invention it is also advantageous for the antibiotic component to contain at least one amino group.

Furthermore, it is advantageous in accordance with the invention that alkyl sulfates, cycloalkyl sulfates, cycloalkylalkyl sulfates, aryl sulfates, alkylaryl sulfates, alkyl sulfamates, cycloalkyl sulfamates, alkylcycloalkyl sulfamates, aryl sulfamates, alkylaryl sulfamates, alkyl sulfonates, fatty acid 2-sulfonates, cycloalkyl sulfonates, cycloalkylalkyl sulfonates, aryl sulfonates and alkylaryl sulfonates with 6 to 30 carbon atoms in each case are preferred as amphiphilic components.

It is advantageous in accordance with the invention that aryl sulfates, alkylaryl sulfates, aryl sulfamates, alkylaryl sulfamates, aryl disulfonates, alkylaryl disulfonates, aryl trisulfonates and alkylaryl trisulfonates built up on the basis of monocyclic, dicyclic, tricyclic, tetracyclic, pentacyclic, hexacyclic, heptacyclic and octocyclic aromatic ring systems are preferred as amphiphilic components.

In accordance with the invention, it is advantageous that cycloalkyl sulfates, alkylcycloalkyl sulfates, cycloalkyl sulfonates, alkylcycloalkyl sulfonates, cycloalkyl sulfonates, alkylcycloalkyl sulfonates, cycloalkyl sulfamates and alkylcycloalkyl sulfamates built up on the basis of monocyclic, dicyclic, tricyclic, tetracyclic, pentacyclic, hexacyclic, heptacyclic and octocyclic saturated ring systems are preferred as amphiphilic components.

Advantageously in accordance with the invention, sodium dodecyl sulfate, sodium tetradecyl sulfate, sodium hexadecyl sulfate, sodium octadecyl sulfate, sodium docosanyl sulfate, sodium dodecyl sulfonate, sodium tetradecyl sulfonate, sodium hexadecyl sulfonate, sodium octadecyl sulfonate, sodium dodecylbenzyl sulfonate and sodium cholesterol sulfate are especially preferred as amphiphilic components.

Furthermore, it is in accordance with the invention that especially allomycin, amicetin, amikacin, ampramycin, bekanamycin, betamicin, butirosin, destomycin, dibekacin, dihydrostreptomycin, flambamycin, fortimycin A, fortimycin B, framycetin, gentamicin, hikizimycin, homomycin, hybrimycin, hygromycin B, kanamycin, kasuhamycin, lividomycin, minosaminoycin, neomycin, netilmicin, paromomycin, parvulomycin, puromycin A, ribostamycin, rimocidin, ristosamine, ristomycin, sagamycin, sisomicin, sorbistin, spectinomycin, streptomycin, tobramycin, tunicamycin, vancomycin, verdamycin from the aminoglycoside antibiotics group are preferred as the antibiotic component.

In accordance with the invention, it is advantageous that clindamycin and lincomycin are preferred as antibiotic components from the lincosamide antibiotics group.

It is advantageously in accordance with the invention that tetracycline, chlorotetracycline, oxytetracycline, demethyl chlorotetracycline, methacycline, doxycycline, rolitetracycline and minocycline are preferred as antibiotic components from the tetracycline antibiotics group.

It is also advantageous in accordance with the invention that the antibiotic component is present in the protonized salt form, whereby chloride ions, bromide ions, hydrogen sulfate ions, sulfate ions, dihydrogen phosphate ions, hydrogen phosphate ions, phosphate ions, acetate ions, succinate ions and lactate ions are preferred as counter-ions .

In accordance with the invention, it is furthermore preferred that 0.01 to 10 constituent parts by mole of the amphiphilic components are mixed with one molar constituent part of the antibiotic components.

It is advantageous in accordance with the invention that the proportion of the delay released antibiotic components to the overall amount of antibiotic components can be determined through the ratio of the amount of amphiphilic constituent parts to the amount of antibiotic constituent parts.

In accordance with the invention, it is also advantageous that anhydrous, organic auxiliary components have hydrolytically cleavable carboxylic acid ester compounds and/or hydrolytically cleavable carboxylic acid amide compounds and/or hydrolytically cleavable carboxylic acid anhydride compounds and/or hydrolytically cleavable phosphoric acid ester compounds and/or hydrolytically cleavable phosphoric acid amide compounds and/or enzymatically cleavable carboxylic acid ester compounds and/or enzymatically cleavable carboxylic acid amide compounds and/or enzymatically cleavable carboxylic acid anhydride compounds and/or enzymatically cleavable phosphoric acid ester compounds and/or enzymatically cleavable phosphoric acid amide compounds.

It is also advantageously in accordance with the invention that oligoester and polyester of L-lactic and/or D-lactic acid and/or 2-hydroxy ethanoic acid and/or 2-hydroxy-ethoxy ethanoic acid and/or 3-hydroxy butyric acid and/or 4-hydroxy butyric acid and/or 4-hydroxy hexanoic acid and 6-hydroxy hexanoic acid, and if need be co-oligo ester and/or co-polyester and if need be ter-oligoester and/or ter-polyester of hydroxy carboxylic acid are used as anhydrous, organic auxiliary components.

It is in accordance with the invention that oligoamides and/or polyamides are used as anhydrous organic auxiliary components which contain amino acids as components.

In accordance with the invention, the amino acids glycine and/or L-alanine and/or D-alanine and/or L-valine and/or D-valine and/or L-threonine and/or D-threonine and/or L-aspartic acid and/or D-aspartic acid and/or L-asparagine and/or D-asparagine and/or L-glutamic acid and/or D-glutamic acid and/or L-glutamine and/or D-glutamine and/or L-omithine and/or D-ornithine and/or L-lysine and/or D-lysine and/or 3-amino propanoic acid and/or R-2-amino butyric acid and S-2-amino butyric acid and/or 3-amino butyric acid and/or 4-amino butyric acid and/or R-2-amino pentanoic acid and/or S-2-amino butyric acid and/or 3-amino pentanoic acid and/or 4-amino pentanoic acid and/or 5-amino pentanoic acid and/or R-2-amino hexanoic acid and/or S-2-amino hexanoic acid and/or 3-amino hexanoic acid and/or 4-amino hexanoic acid and/or 5-amino hexanoic acid and/or 6-amino hexanoic acid and/or R-2-amino heptanoic acid and/or S-2-heptanoic acid and/or 3-amino-heptanoic acid and/or 4-amino heptanoic acid and/or 5-amino heptanoic acid and/or 6-amino-heptanoic acid and/or 7-heptanoic acid and/or R-2-amino octanoic acid and/or S-[2]-octanoic acid and/or 3-amino octanoic acid and/or 4-amino octanoic acid and/or 5-amino octanoic acid and/or 6-amino octanoic acid and/or 7-amino octanoic acid and/or 8-amino octanoic acid and/or R-2-amino nonanoic acid and/or S-2-amino nonanoic acid and/or 3-amino nonanoic acid and/or 4-amino nonanoic acid and/or 5-amino nonanoic acid and/or 6 amino nonanoic acid and/or 7-amino nonanoic acid and/or 8-amino nonanoic acid and/or 9-amino nonanoic acid and/or R-2-amino decanoic acid and/or S-2-amino decanoic acid and/or 3-amino decanoic acid and/or 4-amino decanoic acid and/or 5-amino decanoic acid and/or 6-amino decanoic acid and/or 7-amino decanoic acid and/or 8-amino decanoic acid and/or 9-amino decanoic acid and/or 10-amino decanoic acid and/or 11-amino undecanoic acid and/or L-phenylalanine and/or D-phenylalanine and/or L-tyrosine and/or D-tyrosine and/or L-histidine and/or D-histidine and/or L-tryptophan and/or D-tryptophan are used as building blocks of the oligoamides and polyamides.

In accordance with the invention, advantageously aliphatic alcohols with a number from 12 to 30 carbon atoms are used as anhydrous, organic auxiliary components.

It is furthermore advantageously in accordance with the invention that fatty acids with a number from 12 to 30 carbon atoms are used as anhydrous, organic auxiliary components.

It is also advantageously in accordance with the invention that glycerin tri-fatty acid esters, glycerin di-fatty acid esters and glycerin mono-fatty acid esters are preferred as anhydrous, organic auxiliary components, whereby the fatty acid radicals contain 14 to 22 carbon atoms in each case.

It is advantageously in accordance with the invention that n-alkanes and iso-alkanes with 6 to 30 carbon atoms are preferred as anhydrous, organic auxiliary components.

In accordance with the invention, polyethylene glycol and/or poly propylene glycol with molar masses in the range from 200 to 35,000 are advantageously preferred as anhydrous, organic auxiliary components.

In accordance with the invention, it is advantageous that polyethylene oxide and polypropylene oxide with molar masses in the 35,000 to 1,000,000 range are preferred as organic auxiliary components.

Advantageously in accordance with the invention, gelatine, collagen, cellulose, carboxy methyl cellulose, methyl cellulose, ethyl cellulose, hydroxy ethyl cellulose, propyl cellulose, hydroxy propylcellulose, butyl cellulose, starch, carboxy methyl starch, methyl starch, ethyl starch, hydroxy ethyl starch, propyl starch, hydroxy propyl starch, butyl starch, chitin, carboxymethyl chitin, chitosan, carboxymethyl chitosan, glycogen, carboxymethyl glycogen, alginic acid, alginic acid methyl ester, hyaluronic acid, carboxymethyl hyaluronic acid, cellulose acetate, cellulose proprionate, cellulose butyrate, cellulose sulfate, cellulose phosphate, starch acetate, starch proprionate, starch butyrate,, starch sulfate, starch phosphate, oxidized cellulose, oxidized starch, pullulan, araban, xanthan, guar gum are preferred as anhydrous, organic auxiliary components.

Advantageously in accordance with the invention, anhydrous, organic auxiliary components such as carnauba wax, beeswax, benzoin resin, collophonium and copal resin are preferred.

In accordance with the invention, advantageously polyethylene, polypropylene, polybutadiene, polyisoprene, polychlor butadiene, polymethyl methacrylate, poly-2-hydroxyethyl methacrylate, polymethacrylate, polystyrene, polyvinyl acetate, polyvinyl alcohol, polyvinyl chloride, polyvinylidene chloride, polyvinyl fluoride, polyvinyl pyrrolidone, polytetrafluoroethylene, polycarbonate, polysulfone, polysiloxane and mixtures of these polymers are preferred as anhydrous organic auxiliary components.

In accordance with the invention, acrylic acid esters, acrylic acid amides, methacrylic acid esters, methacrylic acid amides, itaconic acid esters, maleimide and mixtures of them are preferred as anhydrous organic auxiliary components.

In accordance with the invention, it is advantageous that the anhydrous, organic auxiliary component is present in a solid and/or liquid state.

It is advantageously also in accordance with the invention that aryl sulfate, aryl sulfonate, aryl sulfamate and alkylaryl sulfonate are components of a non-cross-linked polymer and/or a cross-linked polymer, whereby polymers from the polystyrene, polymethacrylate, polyacrylate, polyamide or polycarbonate group and/or their co-polymers and/or their ter-polymers are preferred.

It is advantageous in accordance with the invention that calcium hydrogen phosphate, calcium hydrogen phosphate-dihydrate, hydroxyl apatite, fluorapatite, calcium polyphosphate, tricalcium phosphate, tetracalcium phosphate, calcium sulfate, calcium sulfate hemihydrate, calcium sulfate-dihydrate, calcium lactate, sodium hydrogen carbonate, calcium carbonate, magnesium carbonate, calcium hydroxide, magnesium hydroxide, magnesium oxide and mixtures of these substances are used in the form of coarsely dispersed (0.5 to 2 mm) and/or highly dispersed powder as inorganic auxiliary components.

In accordance with the invention, it is advantageous that resorbable glasses, non-resorbable glasses, resorbable glass ceramics, non-resorbable glass ceramics, resorbable ceramics and non-resorbable ceramics are used as inorganic auxiliary components.

It is advantageous in accordance with the invention that at least one antibiotic from the penicillin antibiotics, the cephalosporin antibiotics, the 4-quinolone antibiotics and the macrolide antibiotics or at least one representative of the sulfonamide chemotherapeutic agents are used as biologically active auxiliary components.

Advantageously in accordance with the invention, if need be representatives of the analgesics and/or anti phlogistics agents are used as biologically active auxiliary components.

It is furthermore in accordance with the invention that the salt-like component and the antibiotic component are suspended in the anhydrous, organic auxiliary components and form an injectable suspension.

In accordance with the invention, the antibiotic/antibiotics preparation, especially an injectable suspension, is used as a resorbable implant and/or as a non-resorbable implant.

It is furthermore in accordance with the invention for the molded elements, granulates, powders, tubes, foils, shaped masses and threads to be used as resorbable implants and/or as non-resorbable implants.

It is in accordance with the invention that the molded elements, granulates and powder manufactured on the basis of the antibiotic/antibiotics preparation are plastically moldable and modelable.

It is likewise in accordance with the invention that resorbable implants and non-resorbable implants, especially in the form of molded elements, granulates, powders, foils, tubes, shaped masses or threads are coated with the antibiotic/antibiotics preparation, especially by pressing and/or immersion and/or spraying and/or calendering and/or extrusion and/or sintering and/or melting on.

It is in accordance with the invention that the antibiotic/antibiotics preparation is applied as a coating onto resorbable porous glasses, to non-resorbable porous glasses, to resorbable porous glass ceramics, to non-resorbable porous glass ceramics, to resorbable porous ceramics and to non-resorbable porous ceramics.

Finally, it is in accordance with the invention that the antibiotic/antibiotics preparation is applied as a coating to resorbable plastic implants, to non-resorbable plastic implants and to metal implants.

The object of the present invention is to be explained in greater detail on the basis of the following examples 1-2.

Manufacture of the antibiotic/antibiotics preparation.

### EXAMPLE 1

A mixture of 51 mg of gentamicin sulfate (700 U/mg, Fluka), 51 mg of sodium dodecyl sulfate, 280 mg carnauba wax, 1118 mg calcium sulfate dihydrate (Fluka) and 1 ml of water is prepared and dried after mixing over calcium chloride. The mixture is subsequently ground. In each case, 200 mg of this mixture are pressed with a press at a pressure of 5 tons inside of two minutes to disk-like, firm molded elements with a diameter of 13 mm.

### EXAMPLE 2

A mixture of 51 mg of gentamicin sulfate (700 U/mg, Fluka), 51 mg of sodium dodecyl sulfate, 140 mg beeswax, 1258 mg calcium sulfate dihydrate (Fluka) and 1 ml of water is prepared and dried after mixing over calcium chloride. The mixture is subsequently ground. In each case, 200 mg of this mixture are pressed with a press at a pressure of 5 tons inside of two minutes to disk-like, stable molded elements with a diameter of 13 mm.

Antibiotic release experiments

The molded elements prepared in examples 1 and 2 were introduced into a physiological saline solution and stored in this at 37 C. over a period of twelve days in order to determine the retarded antibiotic release. Sampling took place after 1, 3, 6, 9 and 12 days of storage time. The antibiotics value determination was conducted with an agar diffusion test using Bacillus subtilis ATCC 6633 as a test germ (for results, see Table 1).

TABLE 1
Cumulative gentamicin release from sample elements from examples
1 and 2 as a function of storage time in physiological saline solution
at 37° C.

| | Cumulative gentamicin release (Ma %) | | | | |
|---|---|---|---|---|---|
| | Storage time (d) | | | | |
| Examples | 1 | 3 | 6 | 9 | 12 |
| 1 | 58 | 73 | 84 | 92 | 100 |
| 2 | 51 | 64 | 80 | 92 | 100 |

What is claimed is:

1. Process for manufacturing an antibiotic/antibiotics preparation, comprising mixing water, an amphiphilic component selected from the group of the alkyl sulfates, aryl sulfates, alkylaryl sulfates, cycloalkyl sulfates, alkylcycloalkyl sulfates, alkyl sulfamates, cycloalkyl sulfamates, alkylcycloalkyl sulfamates, aryl sulfamates, alkylaryl sulfamates, alkyl sulfonates, fatty acid-2-sulfonates, aryl sulfonates, alkylaryl sulfonates, cycloalkyl sulfonates, alkylcycloalkyl sulfonates, alkyl disulfates, cycloalkyl disulfates, alkyl disulfonates, cycloalkyl disulfonates, aryl disulfonates, alkylaryl disulfonates, aryl trisulfonates and alkylaryl trisulfonates as well as at least one antibiotic component selected from the group of aminoglycoside antibiotics, lincosamide antibiotics and tetracycline antibiotics, an organic auxiliary component and/or an inorganic auxiliary component and optionally at least one biologically active auxiliary component and optionally shaping into molded elements and or granulates and/or powders and/or foils and/or shaped masses and/or thread.

2. Process for manufacturing an antibiotic/antibiotics preparation according to claim 1, wherein shaping is performed by means of pressing and/or extrusion and/or grinding and/or calendering and/or casting and/or spinning and/or sintering.

3. Process for manufacturing an antibiotic/antibiotics preparation according to claim 1, wherein the antibiotic component contains at least one amino group.

4. Process for manufacturing an antibiotic/antibiotics preparation according to claim 1, wherein the antibiotic component is present in a protonized salt form, whereby chloride ions, bromide ions, hydrogen sulfate ions, sulfate ions, dihydrogen phosphate ions, hydrogen phosphate ions, phosphate ions, acetate ions, succinate ions and lactate ions are used as counter-ions.

5. Process for manufacturing an antibiotic/antibiotics preparation according to claim 1, wherein the ratio of the molar amount of the amphiphilic component to the molar amount of the antibiotic component lies in the 0.01 to 10 range.

6. Process for manufacturing an antibiotic/antibiotics preparation according to claim 1, wherein the organic auxiliary component contains hydrolytically cleavable carboxylic acid ester compounds and/or hydrolytically cleavable carboxylic acid amide compounds and/or hydrolytically cleavable carboxylic acid anhydride compounds and/or hydrolytically cleavable phosphoric acid ester compounds and/or hydrolytically cleavable phosphoric acid amide compounds and/or enzymatically cleavable carboxylic acid ester compounds and/or enzymatically cleavable carboxylic acid amide compounds and/or enzymatically cleavable carboxylic acid anhydride compounds and/or enzymatically cleavable phosphoric acid compounds and/or enzymatically cleavable phosphoric acid amide compounds.

7. Process for manufacturing an antibiotic/antibiotics preparation according to claim 1, wherein the organic auxiliary components is present in the solid and/or the liquid state.

8. Process for manufacturing an antibiotic/antibiotics preparation according to claim 1, wherein the aryl sulfates, aryl sulfonates, aryl sulfamates and alkylaryl sulfonates are components of a non-cross-linked and/or a cross linked polymer, whereby polymers from the polystyrene, polymethacrylate, polyacrylate, polyamide, polycarbonates and/or their copolymers and/or their ter-polymers are used.

9. An antibiotic/antibiotics preparation prepared by a process according to claim 1.

10. An injectable suspension comprising an antibiotic/antibiotics preparation according to claim 9.

11. A resorbable or non-resorbable implant comprising an antibiotic/antibiotics preparation according to claim 9.

12. The implant according to claim 11, which is in the form of molded elements, granulates, powders, foils, shaped masses or threads.

13. The implant according to claim 12, wherein the molded elements, granulates, powders, foils, shaped masses or threads are plastically moldable or modelable.

14. A method of preparing an implant comprising coating an antibiotic/antibiotics preparation according to claim 9 on a resorbable or non-resorbable material.

15. A method of preparing an implant comprising coating an antibiotic/antibiotics preparation according to claim 9 on a material selected from resorbable porous glasses, non-resorbable glasses, resorbable porous glass ceramics, non-resorbable porous glass ceramics, resorbable porous ceramics and non-resorbable porous ceramics.

16. A method of preparing an implant comprising coating an antibiotic/antibiotics preparation according to claim 1 on a material selected from resorbable plastic implants, non-resorbable plastic implants and metal implants.

17. A method of treating a microbial infection in a human or animal comprising treating said human or animal with an antibiotic/antibiotics preparation according to claim 9.
